(19) United States
(12) Patent Application Publication
(10) Pub. No.: US 2016/0114160 A1
(43) Pub. Date: Apr. 28, 2016

(54) ELECTRO-MUSCULAR STIMULATION SYSTEM

(71) Applicant: Alexander R. Scott, Townville, PA (US)

(72) Inventor: Alexander R. Scott, Townville, PA (US)

(21) Appl. No.: 14/813,941

(22) Filed: Jul. 30, 2015

Related U.S. Application Data
(60) Provisional application No. 62/069,080, filed on Oct. 27, 2014.

(51) Int. Cl. A61N 1/36; A61N 1/372; A61N 1/04

(52) U.S. Cl. CPC A61N 1/36003; A61N 1/0452; A61N 1/0484; A61N 1/36021; A61N 1/37247

(57) ABSTRACT

What is presented is an electro-muscular stimulation system for the electric stimulation of certain muscle groups of a user. The system comprises a compression suit, control unit, and battery pack. The compression suit is wearable by the user and comprises an EMS device and an accelerometer. The control unit is in electric communication with the EMS device and the accelerometer. The control unit is configured to receive a data input and convert the data input into an electric-stimulation signal that is sent to the EMS device. The accelerometer is configured to provide feedback data to the control unit. The battery pack is configured to provide power to the EMS device and the accelerometer.

The present application incorporates by reference U.S. Provisional Patent Application Ser. No. 62/069,080 filed on Oct. 27, 2014.

### BACKGROUND

Electro-muscular stimulation (EMS) is a process that applies a very weak current to a muscle (or a group of muscles) to stimulate the muscle by causing rapid contractions. The more a muscle is stimulated, the stronger the contractions will be. EMS is useful for the general exercise of functional muscles to improve muscle tone and strength. For example, with athletes, EMS can be used as a supplement to stimulate certain muscle group before and after conventional conditioning exercises (running, weight training, boxing, etc.). This stimulation can also, in turn, bring additional strength to the supplemented muscle group than would have been provided without the assistance of EMS. EMS can also be used to assist to recondition muscles (or a group of muscles) that have, for whatever reason, lost tone and/or strength, been injured, or are in need to effect cosmetic improvements.

Limitations exist with current EMS providing devices in the field. These EMS devices are typically embodied as pads that are difficult to apply to certain specific muscle groups found on the body. When attempting to apply these EMS providing devices to certain muscle groups, some kind of adhesive is required. These adhesives can be very messy and inconvenient in general. Moreover, they are not mobile and typically have to be connected to a machine that both powers the EMS device and tells it the specific amount of current to apply. Finally, these EMS devices, and the machines in which they are connected, are not able to be programed specifically for certain workouts so as to provide the best conventional conditioning exercise supplement possible. What is presented is a mobile EMS system that is easy to apply to all muscle groups on the body and can be programed as a supplement for specific conditioning exercises.

### BRIEF DESCRIPTION OF DRAWINGS

For a more complete understanding and appreciation of this invention, and its many advantages, reference will be made to the following detailed description taken in conjunction with the accompanying drawings.

FIG. 1 shows a control unit for the advanced electric muscle stimulation system (hereinafter “AEMSS”);

FIG. 2 shows an exploded view of the control unit of FIG. 1;

FIG. 3 shows a battery pack for the AEMSS;

FIG. 4 shows a front view of the compression suit for the AEMSS; and

FIG. 5 shows a back view of the compression suit for the advanced electric muscle stimulation system.

### SUMMARY

What is presented is an electro-muscular stimulation system for the electric stimulation of certain muscle groups of a user. The system comprises a compression suit, control unit, and battery pack. The compression suit is wearable by the user and comprises an EMS device and an accelerometer. The control unit is in electric communication with the EMS device and the accelerometer. The control unit is configured to receive a data input and convert the data input into an electric-stimulation signal that is sent to the EMS device. The accelerometer is configured to provide feedback data to the control unit. The battery pack is configured to provide power to the EMS device and the accelerometer.

The electro-muscular stimulation system may also comprise a sensor configured to monitor various physical characteristics of the user. In certain embodiments, the compression suit comprises a network of flexible tubes that is configured to facilitate the EMS device and the accelerometer to able to electrically communicate with the control unit. In other embodiments, the compression suit is form fitting to the user to promote muscle repair and assist in reducing post-conditioning muscle atrophy and pain. In further embodiments, the compression suit comprises a series of the EMS devices and a series of the accelerometers. In such embodiments, each EMS device of the series of the EMS devices may be positioned to rest on top of the prominent muscle groups of the user when the compression suit is being worn by the user. Moreover, the compression suit may comprise at least 36 EMS devices. In such embodiments, each accelerometer of the series of the accelerometers may also be positioned over the major joints of the user when the compression suit is being worn by the user.

The battery pack may be releasably joined to the compression suit via a pocket and the battery pack may be a lithium-ion battery. The externally-facing surface of the EMS device may also be covered by the compression suit and the user-facing surface of the EMS device may also be in direct contact with the user. The control unit may include a plurality of exercise modes. Moreover, the control unit may comprise; a housing, touch screen, motherboard, and processor. In such embodiments, the touch screen interface is connected to the housing and covered by a layer of transparent material. The motherboard is located within the housing and is in electric communication with the interface. The processor is located on the motherboard and is in electric communication with the motherboard. Furthermore, in such embodiments, the processor, motherboard, and interface work in conjunction to receive the data input from the user and then convert the data input into an electric-stimulation signal that is to be sent to the EMS device. The housing may also be constructed to handle tough and unstable environments. The user may also be a human being.

What is also presented is an electro-muscular stimulation system for the electric stimulation of certain muscle groups of a user. The system comprises a form fitting compression suit, a control unit, and a battery pack. The form fitting compression suit is wearable by the user and comprises a series of EMS devices and a series of accelerometers. The control unit is in electric communication with the EMS device and the accelerometer. The control unit is also configured to receive a data input and then convert the data input into an electric-stimulation signal that is to be sent to an EMS device. The accelerometer is configured to provide feedback data to the control unit. The battery pack is releasably joined to the compression suit via a pocket. The battery pack is also configured to provide power to the EMS device and the accelerometer. Each EMS device of the series of the EMS devices is positioned to rest on top of the prominent muscle groups of the user when the compression suit is being worn by the user. Each the accelerometer of the series of the accelerometers is positioned over the major joints of the user when the compression suit is being worn by the user.

The electro-muscular stimulation system may also comprise a sensor configured to monitor various physical characteristics of the user. In certain embodiments, the compression suit comprises a network of flexible tubes that is configured to facilitate the EMS device and the accelerometer to able to electrically communicate with the control unit. The externally-facing surface of the EMS device may also be covered by the compression suit and the user-facing surface of the EMS device may also be in direct contact with the user. Moreover, in certain embodiments, the control unit may comprise; a housing, touch screen, motherboard, and processor. In such embodiments, the touch screen interface is connected to the housing and covered by a layer of transparent material. The motherboard is located within the housing and is in electric communication with the interface. The processor is located on the motherboard and is in electric communication with the motherboard. Furthermore, in such embodiments, the processor, motherboard, and interface work in conjunction to receive the data input from the user and then convert the data input into an electric-stimulation signal that is to be sent to the EMS device. The housing may also be constructed to handle tough and unstable environments. The user may also be a human being.

### DETAILED DESCRIPTION

Referring to the drawings, some of the reference numerals are used to designate the same or corresponding parts through several of the embodiments and figures shown and described. Corresponding parts are denoted in different embodiments with the addition of lowercase letters. Variations of corresponding parts in form or function that are depicted in the figures are described. It will be understood that variations in the embodiments can generally be interchanged without deviating from the invention.

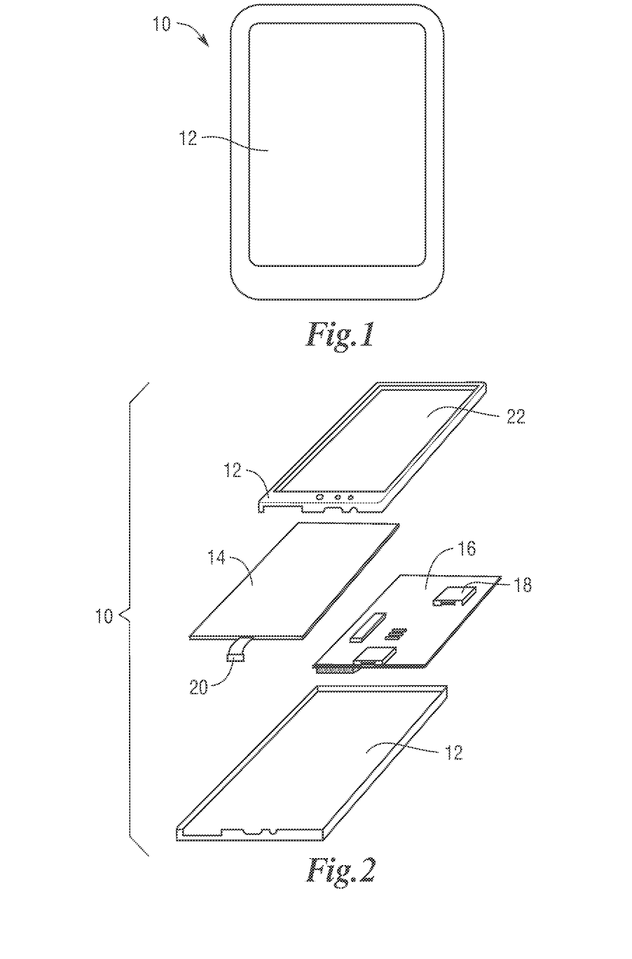

As shown in FIGS. 1 and 2, the AEMSS includes a control unit 10, which is typically a mobile computing device. The control unit 10 has a housing 12, an interface 14, a motherboard 16 and a central processing unit (hereinafter “processor”) 18 located on the motherboard 16 (the interface 14, motherboard 16, and processor 18 each being in electric communication). The control unit 10 also includes a connector 20 that allows the control unit 10 to electrically communicate (connect) to the EMS devices (discussed later). The interface 14 covered by a layer of transparent material 22 on the housing 12 for protective purposes. The interface is typically embodied as a touch screen that incorporates an light emitting diode (“LED”) display or an liquid-crystal display (“LCD”), but may be any display capable for the purposes of the control unit 10. It should be understood that a touch screen interface is not required for the AEMSS and incorporations of an input device (not shown) may be included. In certain instances, the housing 12 may be constructed to certain military specifications to ensure the control unit can handle tough and unstable environments. The connector is typically embodied as a flat-pin connector, such as a 7 way flat pin connector, but may be any connector able to connect to the EMS devices of the AEMSS.

Through the use of the processor 18, motherboard 16, and interface 14, in conjunction, the control unit 10 receives a user's (not shown) data input (typically in the form of user-created instructions made through an exercise module) and converting the data input into an electric-stimulation signal to be sent to the EMS devices of the AEMSS. The control unit 10 makes it possible for electrical stimulation to be provided to specific muscle groups and for certain durations of time, based on the certain exercises of the user. A user is also able to program the control unit 10 for certain pre-programmed exercises (isolated and compound) to maximize the user's conventional conditioning exercise. Through the general implementation of the exercise module (not shown), which is typically embodied as a software application installed on the control unit 10, a user is also capable of ordering the control unit 10 to create a “play list” (a sequential/shuffled list) of pre-programmed exercises that make it possible for electrical stimulation to be provided to specific muscle groups, in a certain pre-programmed order, and for certain durations of time. The data can make adjustments to, but not limited to, the voltage, pulse per second (“PPS”) level, amplitude, polarity, frequency, and waveform of the current being applied to stimulate a specific muscle (or group of muscles). A skilled artisan would see that a user may also be able to program their own unique exercises through the functions of the exercise module.

The control unit 10 also displays feedback data sent to it from a series of accelerometers located on the compression suit (discussed below). In certain embodiments, the control unit 10 can automatically adjust the electric stimulation signal that is to the EMS devices through the feedback data sent from the accelerometers. In other embodiments, activation of the electric stimulation signal may simply be caused by movement of the accelerometers while the user is wearing the compression suit. The processor is typically embodied as a 1.3 GHZ Snapdragon 800 MSM8974 processor (manufactured by the Qualcomm Corporation), but may be any processor capable of converting a user's data input to an electric stimulation signal sent to the EMS devices.

The control unit 10 may be connected to a network, such as the internet, to create netowrk-based support that stores and tracks the progress of the user as well as provides control unit 10 software updates and information. The user could also connect to a specific internet website through the control unit 10 that allows the user to create their own individual user profile. This will allow the user to record their progress and compare that progress with other user profiles on the website. A kill code may be sent to the control unit 10 from the internet that locks the control unit 10 from its functioning, which in turn stops the entire AEMSS from functioning. The kill code is activated when at least one user parameter is not met, such as, but not limited to, the non-payment of the user for continued use of the AEMSS.

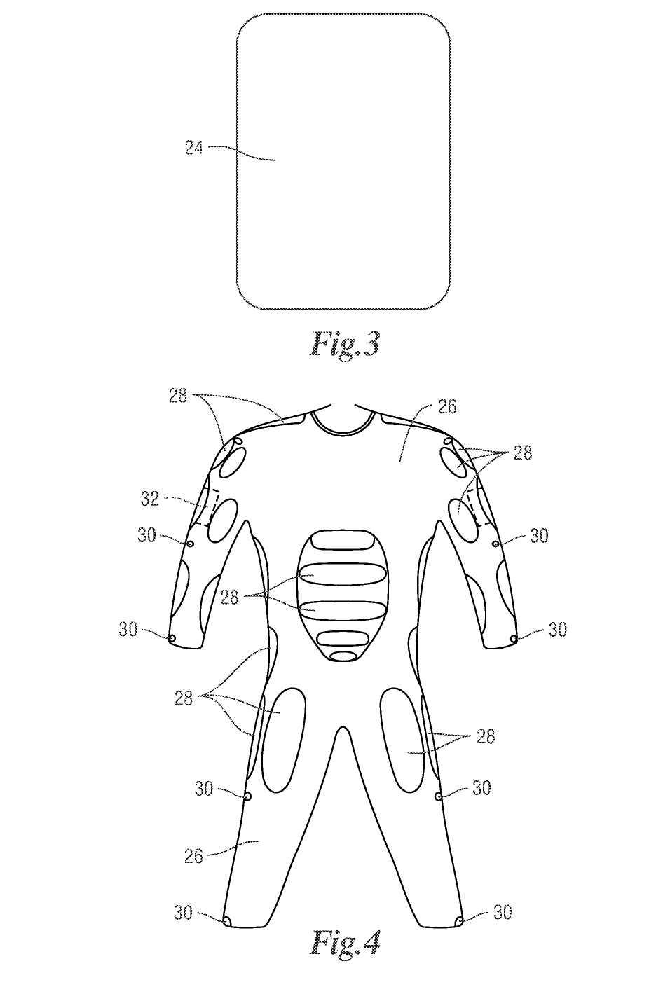

As shown in FIG. 3, a battery pack 24 provides power to the EMS devices and accelerometers of the AEMSS. When the AEMSS is properly constructed, the battery pack 24 is releasably joined at some position on the compression suit. The battery pack 24 usually has a rectangular-plate shape to provide comfort for the user. In certain instances, the battery pack 24 is approximately the same size as the control unit (not shown), but such a size is not required. The battery pack 24 is typically embodied as a lithium-ion battery able to provide power to the EMS devices for extended periods of time and in multiple use sessions without running out of charge. However, any type of battery/device able to provide power to the EMS devices for extended durations of time and in multiple use sessions can be used in the AEMSS.

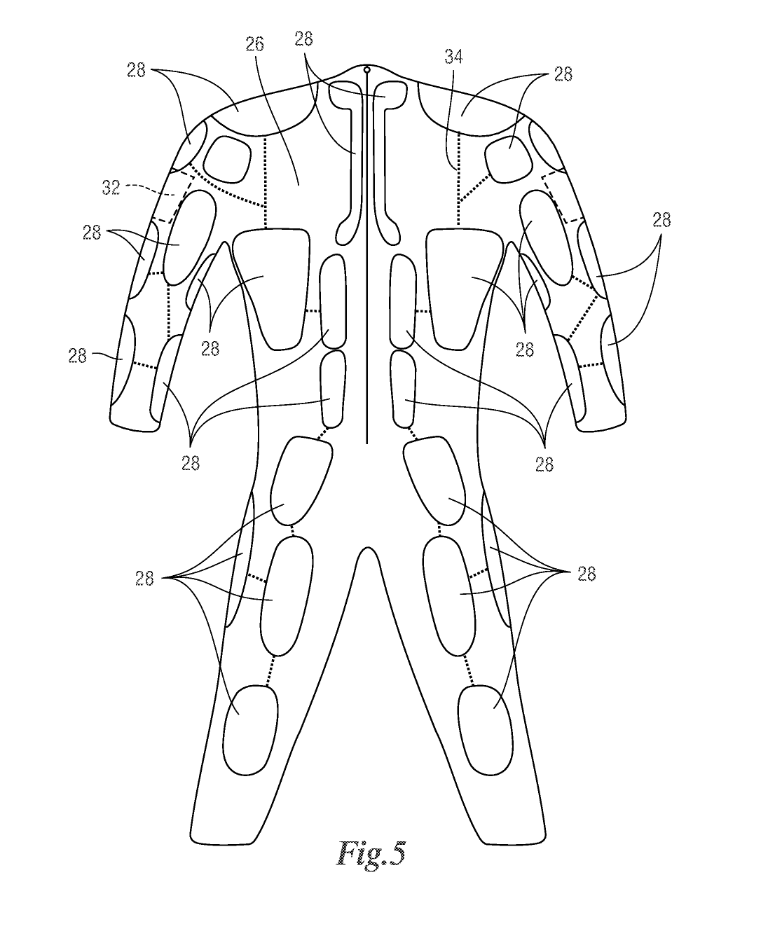

As shown in FIGS. 4 and 5, the AEMSS includes a compression suit 26 equipped with a series of EMS devices 28 and accelerometers 30. The compression suit 26 is form fitting to the user. This form fitting feature promotes muscle repair and assists in reducing post-conditioning muscle atrophy and pain. In this embodiment, the compression suit 26 accommodates at least 36 individual EMS devices 28. In this embodiment, each EMS device is made from a flexible material to form around the muscles (or group of muscles) of the user.

As shown, when properly installed, each EMS device 28 is positioned to rest on top of prominent muscle groups on the body of the user, during use of the AEMSS. However, one having ordinary skill in the art will see that there could be fewer or more EMS devices 28 on the compression suit and these devices do not have to be positioned rest on top of all prominent muscle groups. The compression suit 26 has a series of openings throughout its body. When each EMS device 28 is sewn onto the compression suit 26, the externally-facing surface (i.e. the surface facing away from the user's body) of the EMS device 28 is covered by a layer of the fabric that constructs the compression suit 26 and the user-facing surface of the EMS device 28 is in direct contact with the skin of the user. It should be understood that the EMS devices 28 may be joined to the compression suit 26 in a different manner from the one disclosed herein. In certain embodiments, the EMS devices 28 incorporate a power range between 0.5 volts and 150 volts.

The accelerometers 30 are joined to the compression suit 26 such that they will be positioned over the major joints of the user, during use of the AEMSS. The compression suit 26 also includes a pocket 32 that the battery pack 24 inserts into, during use of the AEMSS. The pocket 32 makes for an easy removal after use of the AEMSS is complete. A network of flexible tubes 34 travels through the entire compression suit 26 makes it possible for both the EMS devices 28 and accelerometers 30 to connect with the connector 20 of the control unit 10.

In certain instances, the AEMSS includes a sensor (not shown) or series of sensors (not shown) that have the ability to monitor various physical characteristics of the user, such as, but not limited to, the user's heart rate, body fat composition, and hydration levels. This sensor, or series of sensors, can be attached to the compression suit 26. They can also be attached to at least one of the EMS devices 28 and/or at least one of the accelerometers 30. They can also be their own independent components in the AEMSS connected to the control unit 10 independently from the other components on the compression suit 26.

In certain instances, the exercise module will be programmed to cause the AEMSS to have two distinct exercise modes. The first mode, the assistance mode, stimulates muscles depending on the specific conventional conditioning exercise the user is conducting. For this mode, the accelerometers 30 provide feedback to the control system (not shown) while the user is exercising. This feedback tells the control system the specific conventional conditioning exercise that is occurring. The control system, alone or through network-based support, will then direct the EMS devices 28 to stimulate the corresponding muscle groups being exercised. This mode is typically used as a warm up function that can be an alternative to stretching prior to the conventional conditioning exercise. The second mode, the isometric workout mode, creates stronger contractions through heavy EMS stimulation. This mode is typically used after conventional conditioning exercise is complete. This mode is similar to traditional EMS stimulation that allows the user to alleviate muscle atrophy and pain.

This invention has been described with reference to several preferred embodiments. Many modifications and alterations will occur to others upon reading and understanding the preceding specification. It is intended that the invention be construed as including all such alterations and modifications in so far as they come within the scope of the appended claims or the equivalents of these claims.

1. An electro-muscular stimulation system for the electric stimulation of certain muscle groups of a user, said system comprises:
a compression suit wearable by the user, said compression suit comprises an EMS device and an accelerometer;
a control unit in electric communication with said EMS device and said accelerometer, said control unit configured to receive a data input and convert the data input into an electric-stimulation signal to be sent to said EMS device, said accelerometer configured to provide feedback data to said control unit; and
a battery pack configured to provide power to said EMS device and said accelerometer.

2. The electro-muscular stimulation system in accordance with claim 1, further comprising a sensor configured to monitor various physical characteristics of the user.

3. The electro-muscular stimulation system in accordance with claim 1, wherein said compression suit comprises a network of flexible tubes configured to facilitate said EMS device and said accelerometer to electrically communicate with said control unit.

4. The electro-muscular stimulation system in accordance with claim 1, wherein said compression suit is form fitting to the user to promote muscle repair and assist in reducing post-conditioning muscle atrophy and pain.

5. The electro-muscular stimulation system in accordance with claim 1, wherein said compression suit comprises a series of said EMS devices and a series of said accelerometers.

6. The electro-muscular stimulation system in accordance with claim 5, wherein each said EMS device of said series of said EMS devices is positioned to rest on top of the prominent muscle groups of the user when said compression suit is being worn by the user.

7. The electro-muscular stimulation system in accordance with claim 6, wherein said compression suit comprises at least 36 EMS devices.

8. The electro-muscular stimulation system in accordance with claim 5, wherein each said accelerometer of said series of said accelerometers is positioned over the major joints of the user when said compression suit is being worn by the user.

9. The electro-muscular stimulation system in accordance with claim 1, wherein said battery pack is releasably joined to said compression suit via a pocket.

10. The electro-muscular stimulation system in accordance with claim 1, wherein the externally-facing surface of said EMS device is covered by said compression suit and the user-facing surface of said EMS device is in direct contact with the user.

11. The electro-muscular stimulation system in accordance with claim 1, wherein said battery pack is a lithium-ion battery.

12. The electro-muscular stimulation system in accordance with claim 1, wherein said control unit includes a plurality of exercise modes.

13. The electro-muscular stimulation system in accordance with claim 1, wherein said control unit comprises:
a housing;
a touch screen interface connected to said housing and covered by a layer of transparent material;
a motherboard located within said housing and in electric communication with said interface;
a processor located on said motherboard and in electric communication with said motherboard; and
wherein said processor, motherboard, and interface work in conjunction to receive the data input from the user and convert the data input into an electric-stimulation signal to be sent to said EMS device.

14. The electro-muscular stimulation system in accordance with claim 13, wherein said housing is constructed to handle tough and unstable environments.

15. The electro-muscular stimulation system in accordance with claim 1, wherein the user is a human being.

16. An electro-muscular stimulation system for the electric stimulation of certain muscle groups of a user, said system comprises:
a form fitting compression suit wearable by the user, said compression suit comprises a series of EMS devices and a series of accelerometers;
a control unit in electric communication with said EMS device and said accelerometer, said control unit configured to receive a data input and convert the data input into an electric-stimulation signal to be sent to an EMS device, said accelerometer configured to provide feedback data to said control unit;
a battery pack releasably joined to said compression suit via a pocket, said battery pack configured to provide power to said EMS device and said accelerometer;
wherein each said EMS device of said series of said EMS devices is positioned to rest on top of the prominent muscle groups of the user when said compression suit is being worn by the user; and
wherein each said accelerometer of said series of said accelerometers is positioned over the major joints of the user when said compression suit is being worn by the user.

17. The electro-muscular stimulation system in accordance with claim 16, further comprising a sensor configured to monitor various physical characteristics of the user.

18. The electro-muscular stimulation system in accordance with claim 16, wherein said compression suit comprises a network of flexible tubes configured to enable said EMS device and said accelerometer to electrically communicate with said control unit.

19. The electro-muscular stimulation system in accordance with claim 16, wherein the externally-facing surface of said EMS device is covered by said compression suit and the user-facing surface of said EMS device is in direct contact with the skin of the user.

20. The electro-muscular stimulation system in accordance with claim 16, wherein said control unit comprises:
a housing;
a touch screen interface connected to said housing and covered by a layer of transparent material;
a motherboard located within said housing and in electric communication with said interface;
a processor located on said motherboard and in electric communication with said motherboard; and
wherein said processor, motherboard, and interface work in conjunction to receive the data input from the user and convert the data input into an electric-stimulation signal to be sent to said EMS device.
